SEQUENCE LISTING (3 sequences)

SEQ ID NO: 1 - DNA, 299 bp, Arabidopsis thaliana
caccaaagcccaaattgcatttaatgagcgctcacaacataatctccaaagagatgtcctttctatgcagttgggtatgaaccccaacaataaatccctttgggcccaacaaccgtttgaacagctgtttggtatgtcaagtgcagaactgaataagagtgaggtgaacactggccagagatcaagtggcatgacaaaggatgattcctcatacacagagtgtgaggcagagcttcttcaatctcttaggctttgcataatgaatatcttgaaactggaaggatcaggagggctctagg

SEQ ID NO: 2 - DNA, 973 bp, Hordeum vulgare
caccaaaggatctgcatggcatggactggcgcttccgccatatcttccgtggtaagtttctgttccgtgccttgctctgatctgtggcagttttacatccccatgtatgcccagtgtgtgtgatctgaagctgataacttcagtagaccatttggttgtagcttgcaatcagtgacaccgcaacacatcaaatctgcctataaattgcaagggtttatcttgtacttgatgatggtgatggcaccgatgataattgtgttaaccgagtgcaaatcaacagggcaacctaggaggcatctccttcagagcggttggagtgtgtttgtcagttccaaaaggcttgtagctggggatgccttcattttcctcaggtctgttgctgtttccttactcaccagcatagcaattcttaacctggtgctaaatgtgttctgctccacacagaggagagagtggcgagcttcgtgttggtgttaggcgggctatgagacagctgtccaacgtgccttcttcagtcatttctagtcatagcatgcatcttggggtccttgcaactgcatggcacgctatcaacacgaaaagcatgttcacggtctactacaaacctaggtacatcaacaatgctaaggcaatcatgcccttctatatgtagtcattaatttgttcctggtggctcattctgagtacttacactactgtctaatctttggtcgattttagaacgagcccttcagagttcattataccatatgatcaatatatggagtctgtgaagaacaactattcaattgggatgagattcaggatgaggtttgaaggcgaagaggcaccagagcaaaggtgactgtcgtaattgcttttcctacaagtgtagtttggtgtgcatgatcccccaacagcaccgagtagatcatttctaatttgctgtttccattgtcatataggtttactggtactatagttggcagtgaaa

SEQ ID NO: 3 - protein, 141 aa, Hordeum vulgare
KDLHGMDWRFRHIFRGQPRRHLLQSGWSVFVSSKRLVAGDAFIFLRGESGELRVGVRRAMRQLSNVPSSVISSHSMHLGVLATAWHAINTKSMFTVYYKPRTSPSEFIIPYDQYMESVKNNYSIGMRFRMRFEGEEAPEQR

(19) United States
(12) Patent Application Publication
(10) Pub. No.: US 2011/0190379 A1
(43) Pub. Date: Aug. 4, 2011

(54) PLANT PATHOGEN RESISTANCE

(76) Inventor: Paul Nicholson, Norfolk (GB)

(21) Appl. No.: 13/061,443

(22) PCT Filed: Sep. 1, 2009
(86) PCT No.: PCT/GB09/51098

§ 371 Date: Apr. 4, 2011

(30) Foreign Application Priority Data
Sep. 1, 2008 (GB) 0815869.3

(51) Int. Cl. A01N 57/24; A01H 1/00; A01H 5/00; A01P 21/00

(52) U.S. Cl. 514/44 A; 435/7.2; 800/283; 800/295

(57) ABSTRACT

The present invention relates to a method for protecting a plant from infection by a pathogen by decreasing the presence of a plant hormone or reducing the responsiveness of a plant to a plant hormone. In particular, the invention related to infection by a necrotrophic pathogen, such as Fusarium Head Blight (FHB) and the plant hormone is selected from ethylene or gibberellin.

### FIELD OF INVENTION

The present invention relates to a method for protecting a plant from infection by a pathogen by decreasing the presence of a plant hormone or reducing the responsiveness of a plant to a plant hormone. In particular, the invention relates to infection by a necrotrophic pathogen, such as Fusarium Head Blight (FHB). The invention also relates to methods for reducing the presence of mycotoxins in a plant, methods for producing and screening for plants with increased pathogen resistance and related uses.

### BACKGROUND OF THE INVENTION

During their lifecycle, plants are susceptible to a broad range of pathogens, including bacteria, viruses, nematodes and fungi. Pathogen infection of crop plants can have a devastating impact on agriculture due to loss of yield and contamination of plants with toxins. Control of pathogen infection is often through pesticides and the benefit of pesticide use is compromised by their environmental impact. To reduce the amount of pesticides used, plant breeders and geneticists have been trying to identify disease resistance loci and exploit the plant's natural defence mechanism against pathogen attack.

Plants have developed a range of defence mechanisms against pathogen attack. Defence mechanisms include induced resistance, which is elicited by microbial invasion or chemical treatments resulting in hypersensitive reaction (HR), systemic acquired resistance (SAR) or induced systemic resistance (ISR). In ISR, upon local infection by a pathogen, plants respond with a signalling cascade that leads to the systemic expression of a broad spectrum and long-lasting disease resistance that is efficient against fungi, bacteria and viruses. SAR is the phenomenon whereby a plant's own defence mechanisms are induced by prior treatment with either a biological or chemical agent (Heil et al., 2002).

It is known that plant genomes comprises diseases resistance (R) genes and interactions between R genes in plants and their corresponding pathogen avirulence (Avr) genes are the key determinants of whether a plant is susceptible or resistant to pathogen attack. Specificity of the interactions between plants and pathogens is a complex phenomenon with a complicated hierarchy of biological organization. Many R genes, which confer resistance to various plant species against a wide range of pathogens, have been isolated. However, the key factors that switch these genes on and off during plant defence mechanisms remain poorly understood. Other genes that play a role in disease resistance are not involved in the primary recognition of the pathogen, but have a role in downstream signalling and hormonal pathways that affect resistance.

Plant hormones have been implicated in regulating disease resistance. Studies of a wide range of host-pathogen interactions have highlighted the role of three plant hormones (salicylic acid (SA), ethylene (ET) and jasmonic acid (JA)) in mediating defence responses to pathogens. The nature of the pathogen and its mode of obtaining nutrient appear to determine which pathway is deployed by the host to counter infection. SA is predominantly associated with resistance towards biotrophic and hemi-biotrophic pathogens and the establishment of systemic acquired resistance (SAR) (Grant and Lamb, 2006). ET and JA, however, appear to be involved in regulating defence mechanisms in response to necrotrophic pathogens and are also required for induced systemic resistance (ISR) promoted by non-pathogenic root-colonizing bacteria (Feys, 2000; Van Wees, 2000; van Loon et al., 2006; Geraats et al., 2007). This is however an oversimplified view as interactions between the pathways are often more complex. For example, while the SA and ET/JA pathways are often antagonistic, instances of cooperative interactions between ET and SA pathways have been reported and ET and JA act antagonistically in response in some plant-insect interactions. In contrast to dicot species, almost nothing is known about the role of ET signalling in defence responses of monocots. The few studies that have been reported centre about the resistance of rice to Magnaporthe oryzae, the cause of rice blast, and indicate that ET signalling is required for resistance to this disease (Singh et al., 2003; 2004).

Ethylene (ET)

The gaseous plant hormone ethylene is known to regulate many physiological and developmental processes in plants, such as seedling emergence, leaf and flower senescence and fruit ripening. A well-known effect of ethylene on plant growth is the so-called ‘triple response’ of etiolated dicotyledonous seedlings. This response is characterized by the inhibition of hypocotyl and root cell elongation, radial swelling of the hypocotyl, and exaggerated curvature of the apical hook.

The committed step in ethylene biosynthesis is the conversion of S-adenosylmethionine into 1-aminocyclopropane-1-carboxylic acid (ACC) by the enzyme ACC synthase (ACS), which can be blocked by aminoethoxyvinylglycine. ACC is converted to ethylene by ACC oxidase (ACO). This reaction is inhibited by cobalt ions or by aminooxyacetic acid.

The signal-transduction pathway of ethylene has been studied in detail and several comprehensive reviews on the ethylene signalling pathway have been published (Guo et al., 2004; Chang et al., 2004; Bleecker et al., 2004).

Many components are involved in ethylene signalling, including negative and positive regulators. A large number of mutants have been identified in the model plant Arabidopsis thaliana (At), thus helping in the dissection of the ethylene signalling pathway. These can be divided into three distinct categories:
1) constitutive triple-response mutants (i.e. ethylene overproduction1 (eto1), eto2, eto3, constitutive triple response1 (ctr1) and responsive to antagonist1 (ran1)/ctr2);
2) ethylene-insensitive mutants (i.e. ethylene receptor1 [etr1], etr2, ethylene insensitive2 (ein2), ein3, ein4, ein5, and ein6); and
3) tissue-specific ethylene-insensitive mutants (i.e. hookless1 (hls1), ethylene insensitive root1 (eir1), and several auxin-resistant mutants).

Ethylene perception can be abolished by compounds such as silver ions, 2,5-norbornadiene or methylpropone.

The role of ethylene in host resistance is complex and appears to differ, depending upon the pathogen, aiding resistance towards some pathogens but increasing susceptibility towards others (Diaz et al., 2002; Bent et al., 2006; van Loom et al., 19992). Seemingly contradictory results about the role of ethylene in conferring pathogen resistance have been reported. Thus, there is no common mechanism by which pathogen resistance is mediated through ethylene. For example, disruption of ethylene signalling in both At and tomato confers increased resistance to Pseudomonas syringae pv. tomato (O'Donnell et al., 2003), but increased susceptibility to Botrytis cinema (O'Donnell et al., 2003).

The involvement of ET/JA signalling pathways in defence against necrotrophs has previously been well documented in studies with dicot species (Glazebrook, 2005). Studies with different pathogens and plant species have revealed different patterns of interaction between ET and JA signalling pathways. Both of the JA and ET pathways are induced by a pathogen which synergistically activates subsequent signal transduction components and the ensuing resistance expressed by the host is believed to be a consequence of this synergistic interaction between the two pathways. The response of Arabidopsis to infection by Botrytis cinerea is one such example (Berrocal-Lobo et al., 2002). There are also cases where only one of the two signalling pathways appears to be activated and the defence response may be either ET- or JA-specific as exemplified by A. brassicicola, for which resistance requires COl1-mediated signalling but not that of the ET pathway (van Wees et al., 2003).

Gibberellin (Gibberellic Acid=GA)

The plant hormone GA is essential for normal growth of plants. GA-deficient mutants of Arabidopsis thaliana exhibit a dwarfed, dark-green phenotype that can be corrected by the application of exogenous GA. GAs form a large family of diterpenoid compounds and the biosynthesis of GA in higher plants can be divided into three stages: (1) biosynthesis of ent-kaurene in proplastids; (2) conversion of ent-kaurene to GA₁₂ via microsomalcytochrome P450 monooxygenases; and (3) formation of C₂₀- and C₁₉-GAs in the cytoplasm. Many genes that encode enzymes crucial in GA biosynthesis have been identified. These enzymes include ent-copalyl diphosphate synthase (CPS) and ent-kaurene synthase (KS), ent-kaurene oxidase(KO) andent-kaurenoic acid oxidase (KAO) GA20-oxidases (GA20ox), and GA 3-oxidases (for review, see Olszewski et al., 2002). Methods for manipulating the biosynthetic pathway of GA are known in the art, for example from WO 2007/135685 hereby incorporated by reference.

GA acts via a group of orthologous proteins known as the DELLA proteins. The Arabidopsis genome contains genes encoding five different DELLA proteins, the best known of which are GAI and RGA. The DELLA proteins are thought to act as repressors of GA-regulated processes, whilst GA is thought to act as a negative regulator of DELLA protein function. GA overcomes the growth-repressive effects of DELLA proteins, by causing a reduction in their nuclear abundance (Fleck and Harberd, 2002, for review, see Richards et al., 2001 and Thomas et al., 2004). Although first identified in Arabidopsis where the dominant mutant gai (GAI=gibberellin insensitive) exhibits a severely dwarf phenotype, the DELLA proteins are now known to regulate the growth of a wide spectrum of higher plants, including maize, wheat, barley and rice (Peng et al., 1999). The Rht alleles in wheat are known as green revolution genes due to their agricultural importance.

Nucleic acids encoding the GAI gene of Arabidopsis thaliana are described in U.S. Pat. No. 6,830,930 hereby incorporated by reference.

Fusarium Head Blight

Head blight (scab) is a devastating disease afflicting cereals worldwide, particularly in USA, Europe and China. Fusarium head blight (FHB) of wheat can be caused by a number of different Fusarium species, including F. culmorum, F. graminearum, F. avenaceum, F. poae, Microdochium nivale and M. majus. The predominant causal agent of Head Blight in the USA and Europe is Fusarium graminearum, teleomorph Gibberella zeae sensu stricto while in China the closely related species F. asiaticum is more prevalent (O'Donnell et al., 2004). F. graminearum appears to behave as a necrotroph when causing head blight of wheat and barley, inducing cell death as soon as it enters into the cytosol of pericarp cells (Jansen et al., 2005).

In addition to yield losses, this disease is of primary concern because of the accumulation of trichothecene mycotoxins, such as deoxynivalenol (DON), also known asvomitoxin, in grain. Trichothecenes are major mycotoxin contaminants of cereals worldwide (Placinta 1997), causing feed refusal, vomiting, diarrhoea and weight loss in non-ruminant animals and posing a health threat to other animals and humans when exposure levels are high (Gilbert, 2000). This threat is exacerbated by the recent shift in the F. graminearum population in the USA towards greater toxin production and vigour (Ward, 2007).

Host resistance is generally recognised as the most appropriate means to control the disease and minimise the risk to consumers of mycotoxins entering the food and feed chains. Two components of FHB resistance are widely recognised: resistance to initial infection (Type I) and resistance to spread within the head (Type II) (Schroeder and Christensen 1963). DON has been shown to inhibit Type II resistance and so enhancing the spread of FHB pathogens within the head (Desjardins, 1990; Bai et al., 2001).

In recent years, considerable advances have been made in understanding the genetic basis of resistance to FHB and a number of genes and quantitative trait loci (QTL) conferring each type of resistance have been reported (Steed et al., 2005 and Cuthbert, 2007). However, largely because of the difficulty of studying this disease, very little is currently understood about the mechanisms involved in resistance or susceptibility. A potential mechanism of resistance is that of DON glycosylation associated with the type II resistance of the variety Sumai 3.

Ever since the initial discovery of the molecules and genes involved in disease resistance in plants, attempts have been made to engineer durable disease resistance in economically important crop plants. Unfortunately, many of these attempts have failed, owing to the complexity of disease-resistance signalling and the sheer diversity of infection mechanisms that different pathogens use. Thus, there is a need for methods that confer pathogen resistance, in particular to FHB, to a plant. The present invention is aimed at addressing this need.

### SUMMARY OF INVENTION

The invention relates to a method for conferring pathogen resistance to a plant by altering the production of a plant hormone or manipulating the plant hormone signalling pathway in said plant. Specifically, the production of the plant hormone is reduced and/or responsiveness of a plant to a plant hormone is reduced. In particular, the pathogen is a necrotrophic pathogen and the plant hormone is selected from ethylene or gibberellin.

In a first aspect the invention thus relates to a method for conferring resistance to Fusarium Head Blight (FHB) to a plant, comprising decreasing the production of a plant hormone in said plant or reducing the responsiveness of said plant to a plant hormone wherein the plant hormone is selected from gibberellin or ethylene.

In another aspect the invention relates to a method for conferring resistance to FHB to a plant, comprising decreasing the production of ethylene in said plant or reducing the responsiveness of said plant to ethylene.

A third aspect relates to a method of reducing the presence of mycotoxins in a plant comprising decreasing the production of ethylene in said plant or reducing the responsiveness of said plant to ethylene.

A fourth aspect of the invention relates to a method for screening for plants which are resistant to FHB comprising identifying a plant with reduced ethylene production and/or reduced responsiveness to ethylene.

In another aspect the invention relates to a method for producing a plant with increased resistance to FHB comprising manipulating components of the ethylene production or signalling pathway. In another aspect the invention relates to a method for conferring resistance to FHB to a plant, comprising decreasing the production of gibberellin in said plant or reducing the responsiveness of said plant to gibberellin.

In another aspect the invention relates to a method for producing a plant with increased resistance to FHB comprising manipulating components of the gibberellin production or signalling pathway.

In another aspect the invention relates to a method for screening for plants that are resistant to FHB comprising identifying a plant with reduced gibberellin production and/or reduced responsiveness to gibberellin.

In another aspect the invention relates to a use of a nucleic acid in the production of a transgenic plant with increased resistance to FHB wherein said nucleic acid encodes a protein involved in the production of a plant hormone or in the signalling pathway of a plant hormone wherein said plant hormone is selected from ethylene or gibberellin.

The invention also relates to a transgenic plant with increased resistance to FHB, in particular F. graminaerum, with reduced production of a plant hormone or a reduction in the signalling pathway of a plant hormone wherein said plant hormone is selected from ethylene or gibberellin.

Finally, the invention relates to a method for conferring resistance to FHB to a plant, comprising generating transgenic plants that carry a mutation in the gene expressing the auxin response factor 2 or wherein said gene is functionally silenced.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

For example, certain embodiments of the invention include the production of a transgenic plant. This can be done by expressing a transgene in a plant using a construct in an expression vector. Methods for making such vectors are known to those skilled in the art. Expression of such a construct may be driven by a constitutive promoter such as the CaMV 35S promoter to achieve overexpression, or by an inducible expression system. Transformation of plants is a well known technique and can be achieved by Agrobacterium transformation or particle bombardment. Other embodiments relate to introducing mutations in certain genes. Again, the techniques for mutagenesis of plants have been described in the literature.

The term manipulation can be understood as interference. Manipulation of a pathway can be by genetic means, such as mutating a gene or silencing a gene or overexpressing a gene in a plant. Manipulation of the pathway can also be by applying an exogenous agent to the plant which affects the pathway.

In a first aspect, the invention relates to a method for conferring resistance to Fusarium Head Blight (FHB) to a plant, comprising decreasing the production of a plant hormone in said plant or reducing the responsiveness of said plant to a plant hormone wherein the plant hormone is selected from gibberellin or ethylene.

In particular, the invention relates to a method for conferring resistance to FHB to a plant, comprising decreasing the production of ethylene in said plant or reducing the responsiveness of said plant to ethylene.

In another aspect, the invention relates to a method for conferring resistance to FHB to a plant, comprising decreasing the production of gibberellin in said plant or reducing the responsiveness of said plant to gibberellin.

According to the methods and uses of the different aspects of the invention described herein, the plant may be a dicotyledonous plant, preferably Arabidopsis thaliana, or a monocot plant. In a preferred embodiment, the plant is a cereal. For example, the plant may be selected from wheat, barley, rice, oat, rye, sorghum or maize. Preferred embodiments relate to wheat and barley.

According to the different aspects of the invention described herein, Fusarium Head Blight is selected from F. culmorum, F. graminearum, F. avenaceum, F. poae, F. asiaticum or Gibberella zeae. In a preferred embodiment, Fusarium Head Blight is F. graminearum. In one embodiment, Fusarium Head Blight is F. graminearum and the plant is wheat.

As shown in detail in the examples, genetic and chemical studies showed that F. gramineaeum exploits the ethylene signalling pathway of both dicotyledonous and monocotyledonous species. DON-induced cell death was reduced in plants impaired in ethylene signalling demonstrating that its phytotoxicity is, at least in part, mediated by this pathway. The dicotyledonous plant species Arabidopsis thaliana has long been used as a model species to study plant-pathogen interaction but translation to monocotyledonous crop species remains an important challenge. The inventors have shown that the ethylene pathway mediates disease resistance in both Arabidopsis and cereal. This model-crop translation thus provides a framework for crop improvement by identifying allelic variation for components of the ethylene signalling pathway in cereal species.

By reducing responsiveness of a plant to a plant hormone is meant interfering with plant hormone signalling.

According to the methods of the invention, resistance may be conferred by decreasing endogenous ethylene production or reducing the responsiveness of a plant to ethylene. Both, ethylene production and/or responsiveness can be altered by genetic manipulation.

Thus, according to the different aspects of the invention, the level of ethylene production can be reduced by manipulating components of the ethylene biosynthesis pathway. For example, genes that encode for an enzyme involved in ethylene production may be mutated or silenced. The pathway of ethylene production is well understood and key components involved in ethylene biosynthesis have been identified. Thus, it is possible to target components of the ethylene production pathway, such as an enzyme or a protein by influencing the activity of said enzyme or a gene encoding therefor, to decrease ethylene production in a plant and thus reduce the level of ethylene present. For example, the conversion of S-adenosylmethionine into 1-aminocyclopropane-1-carboxylic acid (ACC) by the enzyme ACC synthase (ACS), may be blocked by introducing mutations in the gene encoding for ACS. ACC is converted to ethylene by ACC oxidase (ACO). Introducing a mutation in ACO may therefore also result in reduced ethylene production. For example, the Arabidopsis ethylene-overproducer mutants eto2 and eto3 have been identified as having mutations in two genes, ACS5 and ACS9, respectively; these encode isozymes of 1-aminocyclopropane-1-carboxylic acid synthase (ACS), which catalyse the rate-limiting step in ethylene biosynthesis. Another ethylene-overproducer mutation, eto1, is in a gene that negatively regulates ACS activity and ethylene production. The ETO1 protein directly interacts with and inhibits the enzyme activity of full-length ACS5 but not of a truncated form of the enzyme, resulting in a marked accumulation of ACS5 protein and ethylene. ETO1 thus has a dual mechanism, inhibiting ACS enzyme activity and targeting it for protein degradation. This permits rapid modulation of the concentration of ethylene (Wang et al., 2004).

Thus, according to the different aspects of the invention, ethylene production in a plant, such as Arabidopsis or a cereal, may be reduced by mutating or silencing genes involved in the ethylene biosynthesis pathway, including those genes listed above and their homologs and orthologues, for example genes encoding for ACS or ACO. RNA interference (RNAi) is a technique firstly used in plants to silence genes. The technique is well known and can thus be employed to specifically silence a gene involved in ethylene production. Furthermore, ethylene production may be manipulated by (over)expressing negative regulators of ethylene synthesis, such as ETO1. In anther embodiment, a mutant allele of a gene involved in ethylene production may be (over)expressed in a plant.

The gene manipulated may be a gene encoding for an enzyme of the ethylene biosynthesis pathway or a gene encoding for a protein altering the activity of an enzyme of the ethylene biosynthesis pathway.

Furthermore, agents may be used to decrease ethylene production, such as cobalt ions, silver ions, aminooxyacetic acid or aminoethoxyvinylglycine.

In another embodiment of the method for altering ethylene production in a plant, the gene expressing the auxin response factor 2 is mutated or wherein said gene is functionally silenced.

A large number of components of the ethylene signalling pathway in plants are known, based on studies carried out in Arabidopsis and other plants. The first step of ethylene signal transduction is the perception of ethylene by a family of membrane associated receptors. In Arabidopsis, a family of five receptors has been identified: ETR1/ETR2, ETHYLENE RESPONSE SENSOR1 (ERS1)/ERS2 and EIN4. Ethylene binds to its receptors which results in the inactivation of receptor function. In the absence of ethylene, the receptors are in a functionally active form that constitutively activates a Raf-like serine/threonine (Ser/Thr) kinase, CTR1. CTR1 turns off the pathway. Ethylene binding turns off receptor signalling, thus inactivating CTR which releases the pathway from repression. Stopping receptor signalling with ethylene or by genetically knocking out all of the receptors releases the pathway from inhibition. Thus, loss of function mutants of these receptors remove the negative regulator and lead to constitutive ethylene signalling. Gain of function mutants on the other hand lead to a constitutive repression of the pathway leading to ethylene insensitivity allowing the receptor to repress signalling even in the presence of ethylene. For example, the dominant etr1-1 mutant in Arabidopsis is insensitive to ethylene. EIN2, EIN3, EIN5, and EIN6 are positive regulators of ethylene responses, acting downstream of CTR1. EIN2 loss of function mutants are ethylene insensitive blocking ethylene responses completely demonstrating that the EIN2 gene is crucial for ethylene signalling. EIN3 is a transcription factor that regulates the expression of its immediate target genes such as (ERF1). Thus, a transcriptional cascade that is mediated by EIN3/EIN3-like (EIL) and ERF proteins leads to the regulation of ethylene-controlled gene expression.

Components of the ethylene signalling pathway have also been identified in plants other than Arabidopsis, for example in tomato, sugarcane and rice. As shown in table 2, orthologues to ETR1, EIN2 and ETO2 in rice, barley and wheat have been identified.

A summary of the components is shown in table 1 and the pathway is also illustrated in FIG. 5.

TABLE 1
Components of the ethylene signalling pathway in Arabidopsis. For each
component, mutant alleles have been identified conferring different
phenotypes.

| Ethylene response | | Putative role |
|---|---|---|
| mutant in Arabidopsis | Full name | of the protein |
| etr1 | ethylene receptor 1 | Membrane receptor |
| etr2 | ethylene receptor 2 | Membrane receptor |
| ein2 | ethylene insensitive 2 | Transcription factor |
| ein3 | ethylene insensitive 3 | Transcription factor |
| ein4 | ethylene insensitive 4 | Membrane receptor |
| ein5 | ethylene insensitive 5 | |
| ein6 | ethylene insensitive 6 | |
| ctr | Constitutive triple | kinase |
| | response 1 | |
| ers1 | Ethylene response | Membrane receptor |
| | sensor 1 | |
| ers2 | Ethylene response | Membrane receptor |
| | sensor 2 | |
| eil1 | ein3-like | Transcription factor |

TABLE 2
Sequences orthologous to ETR1, EIN2 and ETO2 in rice, barley
and wheat.

| | | | Similarity to species; |
|---|---|---|---|
| Gene | Species | Sequence | over nucleotides (n.t.) region |
| EIN2 | Rice | AY396568 | |
| | Barley | BM816947.1 | 85% similar to rice over 200 n.t. |
| | Wheat | AL816731.1 | 93% similar to barley over 554 n.t. |
| ETR1 | Rice | AF013979.1 | |
| | Barley | BU968606.1 | 91% similar to rice over 634 n.t. |
| | Wheat | BJ236038.1 | 96% similar to barley over 487 n.t. |
| ETO2 | Rice | XM473608.1 | |
| (ACS5) | Barley | BQ468428.1 | 89% similar to rice over 291 n.t. |
| | Wheat | U42336^{a} | 93% similar to barley over 424 n.t. |

As shown here, the At mutants, etr1 (ethylene resistant), ein2 and ein3 (ethylene insensitive), compromised in ethylene perception and signalling, and eto1 and eto2, (ethylene over-producers) all alter the infection response of At to Fg. Inhibition of ethylene perception and signalling by mutation of ETR1, EIN2, or EIN3 significantly increases resistance to Fusarium graminium (Fg), while overproduction of ethylene (eto1 and eto2 mutations) increases susceptibility (FIG. 1a). Chemical modifiers of ethylene pathways, which chemically mimic the genetic mutants, confirm the involvement of ethylene in Fg resistance (FIG. 1b). The inventors have also shown that alteration of ET levels/perception had similar effects on cereal lines.

Thus, according to the invention, responsiveness of a plant to ethylene is decreased by manipulation of the ethylene signalling pathway.

In one embodiment one or more gene(s) encoding a component of the ethylene signalling pathway is silenced or mutated. Said components are selected from receptors, transcription factors and genes encoding said components.

Any modification that leads to an ethylene insensitive mutant is within the scope of the methods of the invention. For example, the gene encoding a component of the signalling pathway is selected from a gene encoding ETR1, ETR2, ERS1, ERS2, EIN2, EIN3, EIN4, EIN5, EIN6 EIL1, CTR or an orthologue or homolog thereof. To reduce ethylene responsiveness, the gene can be silenced if it is a positive regulator, such as EIN2. RNAi has been used to silence the EIN2 gene in wheat (Travella et al., 2006). Mutations can also be introduced in positive regulators of the ethylene pathways which lead to ethylene insensitivity. If the gene product acts as a negative regulator, then a gain of function mutation that results in ethylene insensitivity reduces ethylene responsiveness. For example, the ETR1 gene may be targeted. Thus, any mutation that confers ethylene sensitivity is useful. In one embodiment, the mutation is in ETR1, EIN2 or Ein3. In one embodiment, EIN2 is silenced in wheat or barley.

In another embodiment, reduction of ethylene responsiveness can be achieved by overexpression of a negative regulator or a mutant allele that acts as negative regulator of the ethylene pathway in a plant resulting in reduced ethylene response in the selected, mutated or transgenic plant.

In another embodiment, reduction of ethylene responsiveness can be reduced by agents that reduce ethylene responsiveness, such as silver ions, 2,5-norbornadiene or methylpropone.

Reduction in ethylene signalling and ethylene production can lead to reduced symptoms and disease development of plants infected with FHB. From a food safety perspective, it is critical that there is also a reduction in mycotoxins.

Thus, in another aspect, the invention relates to a method of reducing the presence of mycotoxins in a plant comprising decreasing the production of ethylene in said plant or reducing the responsiveness of said plant to ethylene. In one embodiment, the mycotoxin is a trichothecene mycotoxin, preferably deoxynivalenol (DON).

In another aspect, the invention relates to a method for producing a plant with increased resistance to FHB by manipulating components of the ethylene production or signalling pathway. Said method may comprise mutagenesis or gene silencing. The plant and FHB species may be selected as described herein. Preferably, FHB is F. graminearum. Preferably, the plant is wheat. In one embodiment, FHB is F. graminearum and the plant is wheat. Reduced ethylene production and/or reduced responsiveness to ethylene is indicative of increased resistance to FHB. The plant may comprise an allelic variant of a gene involved in ethylene signalling.

In a further aspect, the invention relates to a method for screening for plants which are resistant to FHB comprising identifying a plant with reduced ethylene production and/or reduced responsiveness to ethylene. The plant and FHB species may be selected as described herein. Preferably, FHB is F. graminearum. Preferably, the plant is wheat. In one embodiment, FHB is F. graminearum and the plant is wheat. Reduced ethylene production and/or reduced responsiveness to ethylene is indicative of increased resistance to FHB.

In another aspect, the invention relates to the use of a nucleic acid in the production of a transgenic plant with increased resistance to FHB wherein said nucleic acid sequence encodes a protein involved in the production of ethylene or in the signalling pathway of ethylene. The nucleic acid may be a mutant allele of a gene involved in the production of ethylene or in the signalling pathway of ethylene. Suitable genes that may be used are described above.

The invention also relates to a transgenic plant with increased resistance to FHB, in particular F. graminaerum, with reduced production of ethylene or a reduction in the ethylene signalling pathway.

The inventor has shown evidence for the involvement of ARF2 in susceptibility to Fusarium in Arabidopsis. Thus, in another aspect, the invention relates to a method for conferring resistance to FHB to a plant, comprising generating transgenic plants that carry a mutation in the gene expressing the auxin response factor 2 or wherein said gene is functionally silenced. Without wishing to be bound by theory, the inventor believes that the mechanism involved in Fusarium resistance may be linked to alteration in auxin signalling and/or ethylene signalling or production. The plant and FHB species may be selected as described herein. Preferably, FHB is F. graminearum.

The invention relates to a method for conferring resistance to FHB to a plant, comprising decreasing the production of gibberellin in said plant or reducing the responsiveness of said plant to gibberellin. In particular, type 2 resistance is increased. Furthermore, resistance to DON is increased.

By gibberellin or GA is meant a diterpenoid molecule possessing biological activity, i.e. biologically active gibberellins. Biological activity may be defined by one or more of stimulation of cell elongation, leaf senescence or elicitation of the cereal aleurone [alpha]-amylase response. There are many standard assays available in the art, a positive result in any one or more of which signals a test gibberellin as biologically active.

In Arabidopsis, the DELLA proteins are encoded by a family of five genes (GIBBERELLIC ACID INSENSITIVE (GAI), REPRESSOR OF ga1-3 (RGA), and three different REPRESSOR OF ga1-3-LIKE genes (RGL1, RGL2, and RGL3).

TABLE 3
GA Response Mutants in Arabidopsis

| | | Role of | |
|---|---|---|---|
| GA Response | | Wild-Type | |
| Mutants in | | Allele in GA | |
| Arabidopsis | Phenotype | Signalling | Predicted Protein |
| gai-1 | GA-insensitive | Negative | Transcriptional |
| | Dwarf | regulator | regulator |
| | (Dominant) | | |
| gai-t6 | GA- | Negative | Transcriptional |
| | independent | regulator | regulator |
| | growth | | |
| | (Recessive) | | |
| gar2-1 | GA- | Not clear | Not clear |
| | independent | | |
| | growth | | |
| | (Dominant) | | |
| pkl | Dwarf with | Positive | CHD3 chromatin |
| | reduced | regulator | remodeling factor |
| | GA response | | |
| | (Recessive) | | |
| rga | GA- | Negative | Transcriptional |
| | independent | regulator | regulator |
| | growth | | |
| | (Recessive) | | |
| rga-Δ17 | GA-insensitive | Negative | Transcriptional |
| | Dwarf | regulator | regulator |
| | (Dominant) | | |
| shi | GA-insensitive | Negative | RING finger |
| | dwarf | regulator | protein |
| | (Dominant) | | |
| sly1 | GA-insensitive | Positive | F-box protein |
| | dwarf | regulator | |
| | (Recessive) | | |
| Spy | GA- | Negative | OGT (O-linked N- |
| | independent | regulator | Acetylglucosamine |
| | growth | | transferase) |
| | Recessive | | |

Mutants have also been identified in other species, in particular cereals and the corresponding genes, which are orthologues of the Arabidopsis genes, have been identified. Rht-B1b and Rht-D1b in wheat are semidominant altered function mutant alleles of the Rht-1 height regulating genes and orthologues of the Arabidopsis GAI gene. GAI orthologues also include D8 in maize, SLR and GID in rice and SLN in barley. Rht genes (Rht1 and Rht2) in wheat have been used to produce the semi-dwarf varieties of the Green Revolution. Extreme dwarf varieties carry Rht3 or Rht10. RHT genes are reduce GA responsiveness, interfering with GA signalling. DELLA mutants, such as Rht1, Rht2 and Rht3 fail to respond to GA and continue to restrain growth even when GA is present.

In one embodiment, gibberellin signalling is decreased. This may be done by manipulation of the gibberellin signalling pathway. For example, one or more gene encoding a component of the gibberellin signalling pathway may be silenced or mutated. Genes involved in GA signalling in a number of plants are known and have been characterised. Thus, one of the genes listed in table 3 or a homolog or orthologue thereof may be silenced or mutated. Specifically, one of the following genes may be silenced or mutated: GAI, D8, SL, GID, SLN, RHT1, RHT2 and RHT3 or a homolog or orthologue therefore. Specifically, if the method is applied to wheat, one of the following genes can be mutated or silenced: RHT1, RHT2 and RHT3. In barley, SLN may be targeted. In rice, GID or SLR may be targeted.

In another embodiment, a gene mutated in the DELLA region is (over)expressed in a plant.

In one embodiment, gibberellin production is decreased. This may be done by manipulation of the gibberellin biosynthesis pathway. For example, one or more gene encoding a component of the gibberellin biosynthesis pathway may be silenced or mutated. Genes involved in GA biosynthesis are known and characterised. Thus, according to the invention, the gene targeted may be selected from a gene encoding for one of the following enzymes: copalyl diphosphate synthase; ent-kaurene synthase; Dwarf3; gibberellin 20-oxidase;) gibberellin 7-oxidase; gibberellin 3 [beta]-hydroxylase; ent-kaurene oxidase or a homolog or orthologue thereof. In another embodiment, gibberellin levels may be inhibited or controlled by preparation of an expression construct capable of expressing a RNA or protein product which suppresses the gibberellin biosynthetic pathway sequence, diverts substrates from the pathway or degrades pathway substrates or products. The sequence is preferably a copalyl diphosphate synthase sequence, a 3beta-hydroxylase sequence, a 2-oxidase sequence, a phytoene synthase sequence, a C20-oxidase sequence, and a 2beta, 3beta-hydroxylase sequence.

In another embodiment, the method comprises exposing said plant to an agent reducing gibberellin production. Gibberellin synthesis inhibitors are known which act at different sites in the biosynthetic pathway of gibberellins. Agents which act relatively late in the synthetic pathway are known as Class A gibberellin biosynthesis inhibitors. These include the compounds paclobutrazol and flurprimidol (sold under the trade names Trimmit® and Cutless®, respectively). Class B gibberellin biosynthesis inhibitors, such as Trinexapac-ethyl (Primo®) act relatively early in the gibberellin biosynthesis pathway. For example, the agent may comprise a combination of trinexapac-ethyl with either or both of flurprimidol and paclobutrazol.

In another aspect, the invention relates to a method for screening for plants that are resistant to FHB comprising identifying a plant with reduced gibberellin production and/or reduced responsiveness to gibberellin. In a further aspect, the invention relates to a method for producing a plant with increased resistance to FHB comprising manipulating components of the gibberellin production or signalling pathway. The plant may comprise an allelic variant of a gene involved in gibberellin signalling.

In another aspect, the invention relates to the use of a nucleic acid in the production of a transgenic plant with increased resistance to FHB wherein said nucleic acid encodes a protein involved in the production of gibberellin or in the signalling pathway of gibberellin. The nucleic acid may be a mutant allele of a gene involved in the production of gibberellin or in the signalling pathway of gibberellin. As described above, the nucleic acid may be a mutant allele of a DELLA gene. Thus, said nucleic acid may carry one or more mutations compared to the wild type gene.

The invention also relates to a transgenic plant with increased resistance to FHB, in particular F. graminaerum, with reduced production of gibberellin or a reduction in the gibberellin signalling pathway.

### FIGURES

FIG. 1. Assessment of Arabidopsis ET signalling mutants for resistance to G. zeae. (A), representative disease symptoms on leaves of mutant and parent plants following inoculation with G. zeae 6 dpi. (B), disease severity (6 dpi); (C), conidial production (6 dpi). Data from six independent experiments are presented with standard error.

FIG. 2. Effect of reduced ethylene perception (silver ions) or enhanced ethylene levels on disease symptoms and conidial production following inoculation of Arabidopsis, wheat and barley leaves with G. zeae. Arabidopsis disease symptoms (A), disease severity scores (B), conidial production(C); wheat disease symptoms (D), conidial production (E); barley disease symptoms (F), conidial production (G). Petioles (Arabidopsis) or cut leaf ends (wheat and barley) embedded in agar (control), agar amended with silver thiosulphate (silver) or leaves exposed to ethylene (ethylene).

FIG. 3. Disease symptoms on wheat heads and DON mycotoxin accumulation in grain of wheat differing in gene silencing of Ein2 following spray or point inoculation with G. zeae. Disease score (AUDPC) (A), and DON content of grain (C) following spray inoculation. Disease score (number of infected spikelets) (B), and DON content of grain (D) following point inoculation. Bobwhite, parental line; 1A, transformed line not exhibiting gene silencing; 37A, transformed line exhibiting marked silencing of Ein2.

FIG. 4. Cell death in leaves of wild-type and Ein2 gene-silenced lines in response to DON mycotoxin. (A) Trypan Blue staining revealing cell death in leaves of Bobwhite, parental line (images on left); 37A, transformed line exhibiting marked silencing of Ein2 (images on right). (B) size of areas of cell death about DON inoculation points in Bobwhite and A37.

FIG. 5. Outline of ethylene signalling components (as determined for studies in A. thaliana). ERS1, ERS2, ETR1, ETR2 and EIN4 constitute the group of ethylene receptors. CTR1 is a negative regulator of E/N2, which in tern regulates expression of EIN3. The stability of EIN3 is influenced by EBF1 and EBF2. E/N3 regulates expression of ERF1 (and other ERFs). ERFs influence expression of ACS genes that encode the enzymes in the penultimate step of ethylene biosynthesis. ETO1 represses expression of ACS gene family members, mutation within this gene resulting in higher levels of ethylene production. Abbreviations: ACS=ACC synthase; CTR=constitutive triple response; EIN=Ethylene insensitive; ERS=ethylene response sensor; ETO=ethylene over producer.

FIG. 6. Effect of DON on Arabidopsis germination, DON inhibits seed germination.

FIG. 7. Effect of DON on Arabidopsis germination, GA can reverse the inhibitory effect of DON on seed germination.

FIG. 8. Rht3 confers a significant resistance to F. culmorum (DON-producer) spread and to treatment with DON mycotoxin A) Point inoculation, B) DON treated.

FIG. 9. Response of Maris Huntsman Rht-isogenic lines to point inoculation with DON toxin, Maris Huntsman DON injection, damaged spikes.

FIG. 10. Response of Maris Huntsman Rht-isogenic lines to Petri-tox assay, mean relative DON response in roots.

FIG. 11. DON tolerance (Petritox assay) of GA-insensitive (M640) and GA-sensitive (M463) barley mutants of variety Himalaya (root elongation). Although this is the result of a single test the GA-insensitive mutant showed greater tolerance to DON than the other two genotypes.

FIG. 12. Spreading of necrosis in a) Himalaya barley and b) Rht mutant (line M640).

### EXAMPLES

The invention is further illustrated in the following non limiting examples.

### Example 1

### The Effect of Ethylene on Pathogen Resistance

The dicotyledonous plant Arabidopsis thaliana has long been used as a model species to unravel the molecular basis of host-pathogen interactions, but the translation of results to monocotyledonous crop species such as wheat is yet to be demonstrated. We recently developed an in vitro assay to study interactions between Arabidopsis and F. graminearum and its virulence factor DON (Chen, 2006). We examined mutants compromised in ET, JA and SA pathways for their response to F. graminearum. We present results to show that ET signalling compromises resistance of Arabidopsis to F. graminearum in our assays, resulting in significantly increased colonisation and conidial production. These results were confirmed for Arabidopsis by altering levels and perception of ethylene. We then showed that alteration of ET levels/perception had similar effects on infection of wheat and barley leaves. Wheat lines compromised, by RNA interference gene silencing, for expression of Ethylene Insensitive 2 (Ein2) demonstrated that F. graminearum exploits ethylene signalling to colonise heads and leaves. Our data suggest that F. graminearum exploits ethylene signalling when colonising both dicotyledonous and monocotyledonous plant hosts.

Materials and Methods

Maintenance and Preparation of Inoculum

The F. graminearum inoculum used in all experiments was ‘UK1’, a DON-producing isolate held in the culture collection of the John Innes Centre. Maintenance and preparation of inoculum was done as described in Chen et al. (2006). The concentration of the inoculum used for inoculation was 5×10⁵ conidia ml⁻¹.

Plant Materials and Growth Conditions: Arabidopsis

The Columbia (Col-0) ecotype is the genetic background of all the Arabidopsis plants used unless otherwise stated. The ethylene insensitive mutants etr1-1 and ein2-1 were obtained from Dr. G. Loake (University of Edinburgh). Other lines used in this study were obtained from the Nottingham Arabidopsis Stock Centre (NASC). Plants were grown in a climatically controlled chamber with a relative humidity (RH) of 80% under 16 h/8 h light/dark cycle at 22° C. Leaves from 3 week-old plants were used in all experiments.

Inoculation, Incubation and Assessment of Disease Symptoms: Arabidopsis

A detached leaf infection bioassay system was used as described in Chen et al., (2006). Briefly, rosette leaves were excised and wounded by puncture and the petiole embedded into 0.7% autoclaved and solidified water agar in square (10×10 cm) clear plastic plates with the leaf blade not touching the agar surface. Conidial suspension (5 μl) amended with 75 μM DON was deposited onto the fresh wound on the adaxial leaf surface. The plates were sealed with Parafilm to maintain 100% RH and incubated under 16 h/8 h light/dark cycle at 22° C. Evaluation of disease severity and quantification of conidial production were performed 6 dpi as described by Chen et al., (2006).

Chemical Feeding Treatments: Arabidopsis

A stock solution of SA (100 mM) was prepared in ethanol and was added to cooled 0.7% autolaved water agar in 1:500 (v/v) to give a final concentration of SA in 200 μM. Similarly, a stock solution of silver thiosulphate(50 mM) was prepared with sterile distilled water (SDW), and filter sterilised through 0.2-μm pore filters. The solution was added to 0.7% autolaved water agar in 1:500 (v/v) to give a final concentration 100 μM. To enhance ET levels, 500 mM acidic Ethephon (pH2-3) was freshly prepared and mixed with an equal amount of basic SDW (pH11, by addition of NaOH) and mist sprayed on to the inner surface of the plate lid. Under alkaline conditions Ethephon breaks down to form ethylene, hydrochloric acid, and phosphoric acid. The inoculated leaves are exposed only to the gaseous ethylene. Plates were sealed with Parafilm to maintain 100% RH and incubated as above. In all cases other than treatment with SA, agar was amended with 0.2% ethanol to enable comparison with plates containing SA.

Chemical Feeding Treatments: Wheat

Wheat (Hobbit ‘sib’) and Barley (Golden promise) were grown in 7 cm pots containing John Innes No. 2 compost adjusted to pH 8.0, in controlled environment cabinets under 16 h/8 h 15° C./12° C. day/nights with 70%. For detached leaf assays, 5 cm sections of the central portion of leaf 2 were taken after approximately 2-3weeks (GS12), (Zadoks et al., 1974). Sections were wounded in two positions 2cm from each cut end at opposite sides of the mid rib using gently applied pressure with a glass Pasteur pipette. Leaf sections were placed in 10 cm square plastic boxes containing 1% water agar. Sections were suspended above a well removed from the agar and the cut ends of leaf sections were sandwiched with a slice of the excised agar. Where appropriate the agar was amended with silver ions using silver thiosulphate to 150 μm for the silver treatment. Ethylene levels were raised by the addition of ethephon 50 mM (pH8) to the well beneath the leaf samples in the ethylene treatment plates. Leaves were inoculated at the wound sites with 5 μl of F. graminearum conidia (1×10⁶ conidia m⁻¹) and plates were returned to the growth chamber for 7 days. Conidia were removed by washing sections in 10 ml water (0.05% Tween) for 1 hour. Conidia were harvested by centrifugation, re-suspended in 0.5 ml water, and counted using a haemocytometer.

Inoculation, Incubation and Assessment of Disease Symptoms on Detached Wheat Heads

Spring wheat (Paragon) was grown in 1 L pots containing John Innes No. 2 compost adjusted to pH 8.0, in an unheated glasshouse. At mid-anthesis (GS 65), wheat stems were removed, held in water and submerged ends cut at the peduncle above the terminal node. Cut heads were placed in 15 ml tubes containing water (control) or water amended with 1.5 mM silver nitrate. Heads were inoculated with conidia of F. graminearum as described by Steed et al. (2005), and inoculated heads placed in propagators within controlled environment cabinets under 16 h/8 h light/dark at 15° C./12° C. For each treatment 9 heads were assessed and the experiment was repeated three times. AUDPC was calculated on the basis of visual disease symptoms assessed at 5, 7 and 9 days post inoculation (dpi).

Effect of Ein2 Silencing on FHB Resistance and DON Accumulation in Wheat

Wheat lines were grown as described above for chemical feeding experiments. Two independent experiments were conducted in separate controlled environment cabinets to assess separately Type II (point inoculation) and Type I+II (spray inoculation) resistance in Bobwhite and the transgenic Ein2 RNAi lines A37 and Al. Heads of each line were inoculated at mid-anthesis (GS 65) with conidia of F. graminearum as described by Steed et al. (2005). For each line, between 3 and 9 ears per replicate were assessed with a total of 4 replicates per experiment. Each experiment was repeated twice. Visual disease symptoms were assessed at 7 day intervals for 28 days post inoculation (dpi). Spray-inoculated ears were scored as percentage of spikelets showing disease symptoms and converted to area under the disease progress curve (AUDPC). Point inoculated ears were visually scored for number of infected spikelets 21 dpi. Data from the two independent experiments of both the spray and point inoculation trials were not significantly different and so data from the two experiments were combined for presentation. At harvest, all inoculated heads were hand threshed to retain all kernels. Kernels from each replicate were milled and the flour analysed for DON content using a competitive enzyme immunoassay kit (R-Biopharm, Germany) according to manufacturer's instructions.

Inoculation of Wheat Leaves with DON

Wheat lines were grown as described above and leaf 3 was removed at GS13 and sections prepared as above for the detached leaf assay. Droplets (5 μl) of DON (150 μm) dissolved in water, were applied to the wounded areas. Four replicate plates were used for each line with 6 leaf sections per replicate and a total of 12 lesions per replicate. Plates were incubated for 6 days at 22° C. 16 h/8 h day/night before trypan blue staining. Leave sections were cleared by incubation in 60% ethanol at 70° C. for 1 hour followed by 24 hours at 22° C. Sections were stained in 0.1% trypan blue for 48 hours (0.1% Trypan blue in 1:1:1 lactic acid:glycerol:water), and de-stained in Chloral Hydrate 2.5 g/ml. Lesion areas were analysed using ImageJ free software.

Statistical Analysis

The disease severity, conidial production, DON accumulation and lesion area data were analysed by generalised linear modelling (GLM) using the software package GenStat release 8.1 as reported in Chen et al. (Chen 2006). Individual treatments were compared to controls using the unpaired T-test within Genstat.

Effect of Ein2 Silencing on FHB Resistance in Barley

We have used the sequence for EIN2 to develop a silencing construct for barley. This sequence is shown as SEQ ID No 1. We have inserted this sequence into a construct termed pBract207 for gene silencing of barley. We have transformed 12 barley lines with the EIN2 silencing construct.

Effect of ARF2 Knockout in Arabidopsis and Barley

Our work with Arabidopsis revealed a mutant that is highly resistant to Fusarium graminearum. The sequence of ARF2 is known, the GenBank information is as follows:

| | |
|---|---|
| LOCUS | NM_203251 3396 bp mRNA linear |
| DEFINITION | Arabidopsis thaliana ARF2 (AUXIN |
| | RESPONSE FACTOR 2); protein binding/transcription |
| | factor (ARF2) mRNA, complete cds. |
| ACCESSION | NM_203251 |
| VERSION | NM_203251.2 GI: 145362701 |
| SOURCE | Arabidopsis thaliana (thale cress) |

We have shown that this is a new allele of auxin response factor 2 (ARF2). Loss of function of ARF2 leads to resistance to Fusarium in Arabidopsis. We have constructed barley lines in which it is expected that expression of the ARF2 gene has been silenced/knocked down. These lines are analysed for resistance to Fusarium graminearum. The full barley ARF2 nucleic acid sequence of the construct and the deduced amino acid coding determined by reference to the ARF2 of rice are shown as SEQ ID2 and 3 respectively.

Work by others (Okushima et al 2005) showed that expression of three ethylene biosynthesis genes (ACC synthase—ACS2, ACS6 and ACS8) is impaired in the developing siliques (seed pods) of Arabidopsis. This provides a link between auxin signalling via ARF2 to ethylene biosynthesis (ACS2, ACS6 and ACS8).

We have shown that mutation of another member of the ASC gene family (ACS5) confers resistance of Arabidopsis leaves to Fusarium.

Results

ET Signalling Plays a Role in Enhancing Susceptibility of Arabidopsis to F. graminearum

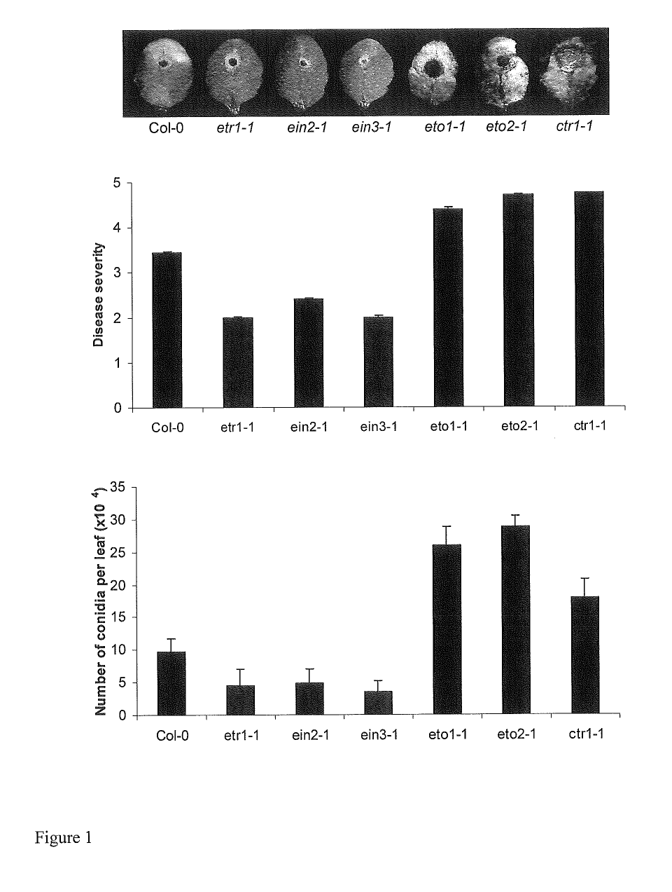

The ET signalling pathway plays an important role in defence against necrotrophic pathogens such as B. cinerea and F. oxysporum (Berrocal-Lobo et al., 2002; 2004). We examined the ethylene insensitive mutants etr1-1, ein2-1 and ein3-1 for their resistance to F. graminearum to determine whether ethylene perception and signal transduction play a role in resistance to this pathogen. All three mutants showed negligible disease or symptoms restricted to the inoculation site, at the time of termination of experiments (6 dpi) when conidial production was assessed (FIG. 1A and 1B). In addition to reduced disease severity, levels of conidial production were also severely and significantly reduced relative to the control (Col-0) (FIG. 1C). Similar results were obtained following inoculation of detached Arabidopsis flowers with conidial production being significantly less on ein2-1 than for Col-0 (P=0.047). These results suggest ET signalling is implicated in the susceptibility of Arabidopsis to F. graminearum. According to this model, we predicted that mutants with constitutive ET signalling would exhibit enhanced susceptibility. To test this, we assessed additional lines with mutations relevant to ET signalling such as the constitutive triple response mutant ctr1-1, those regulating ET biosynthesis such as eto1-1 and those involved in ET biosynthesis such as eto2-1. The conversion of S-adenosylmethionine (AdoMet) to 1-aminocyclopropane-1-carboxylic acid (ACC) by 1-aminocyclopropane-carboxylate synthase (ACS) is deemed to be the rate limiting step in ET biosynthesis. The ACS5 protein is stabilised in the ET biosynthesis mutant eto2-1, leading to enhanced ET production (Vogel et al., 1998). As anticipated, eto1-1, eto2-1 and ctr1-1 all showed significantly enhanced susceptibility to F. graminearum as indicated by more rapid lesion development and extensive fungal growth on the leaves compared to the wild-type Col-0 (FIG. 1A). Taken together these findings indicate that both ET biosynthesis and signalling function to increase the susceptibility of Arabidopsis to infection by F. graminearum.

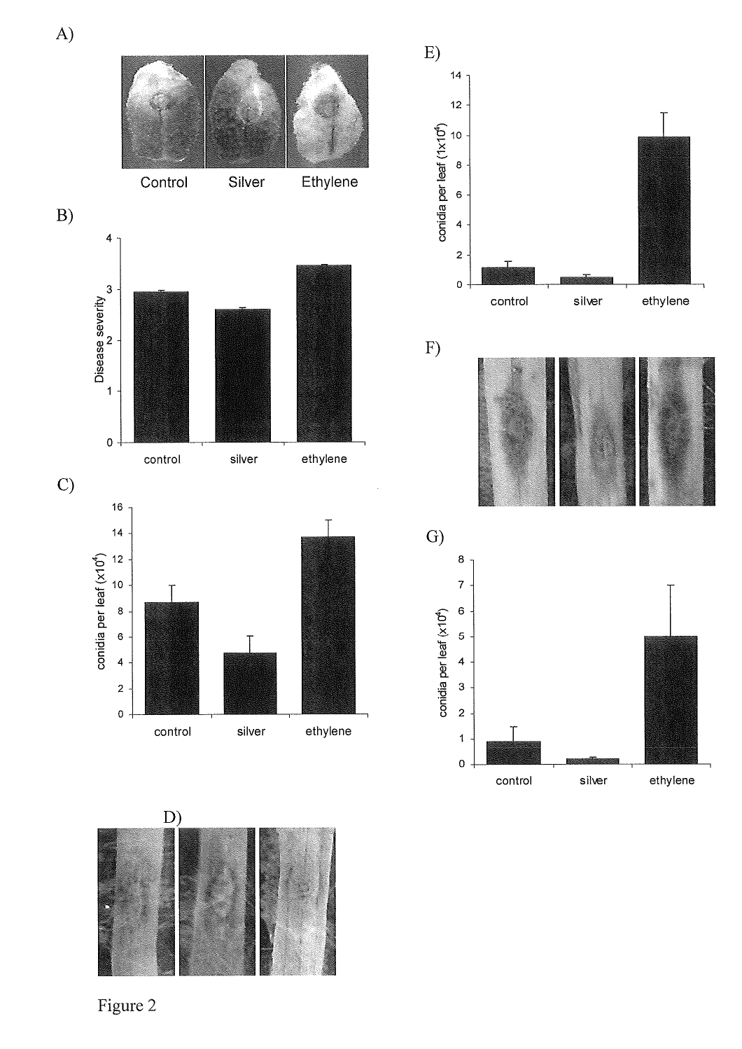

The experiments with the lines containing genetic mutations in the ET pathway suggest an important role of ET in supporting disease development. We then tested whether perturbation of ET levels/signalling would yield similar results. This was achieved by exposing inoculated leaves to raised levels of ethylene (released from Ethephon) or to silver ions, a potent inhibitor of ET perception. As expected, exposure of Col-0 to raised levels of ethylene enhanced susceptibility in respect of both disease severity and conidial production while treatment with silver thiosulphate enhanced resistance (FIGS. 2A, 2B and 2C). To test whether ET accumulation is sufficient or requires a fully functional ET signal transduction for the promotion of susceptibility, we compared the resistance of etr1-1 and ein2-1 and Col-0 to challenge by F. graminearum in the presence and absence of enhanced levels of ethylene. Exposure to ET enhanced disease severity to a similar extent in Col-0 and in the ethylene insensitive mutants etr1-1 and ein2-1 (data not shown), suggesting that ethylene itself, is able to promote susceptibility. However, the overall levels of disease severity in etr1-1 and ein2-1 were significantly lower than in Col-0, suggesting that, in addition to ET accumulation, the functioning of the ET signalling pathway is required for full susceptibility.

ET Signalling Plays a Role in Enhancing Susceptibility of Wheat and Barley Leaves to F. graminearum

The results from conventional genetic and chemical genetic studies show that ET accumulation and signalling promote susceptibility of Arabidopsis following infection by F. graminearum, increasing the rate of colonisation and disease development. We then used a similar chemical genetic approach to determine whether F. graminearum also exploits ET signalling when colonising monocot crop species such as wheat and barley. Increasing ethylene levels or interfering with ethylene perception did not markedly alter lesion size following inoculation of leaves of wheat or barley although the lesion margins tended to be more distinct when ET perception was compromised (FIGS. 2D and 2F). Conidial production was, however, significantly influenced for both wheat and barley. Exposure to enhanced levels of ET led to significantly greater conidial production (P=0.05 and P=0.005) for wheat and barley respectively while conidial production was significantly reduced (P=0.003 and P=0.043) respectively when ET perception was reduced (FIGS. 2E and 2G). The results using detached tissues of wheat and barley indicated that exposure to raised ET levels enhanced fungal colonisation while reduced ET perception decreased fungal colonisation in a manner broadly similar to that seen in Arabidopsis. Following spray inoculation of cut wheat heads bleaching symptoms typical of FHB developed on heads with peduncles immersed only in water. In all three experiments disease development was slower on heads with peduncles immersed in water containing silver ions (1.5 mM) (12-88% reduced). Although the reduction in symptoms differed markedly between experiments, silver-treated heads had significantly lower AUDPC than water treated heads across experiments (P<0.001).

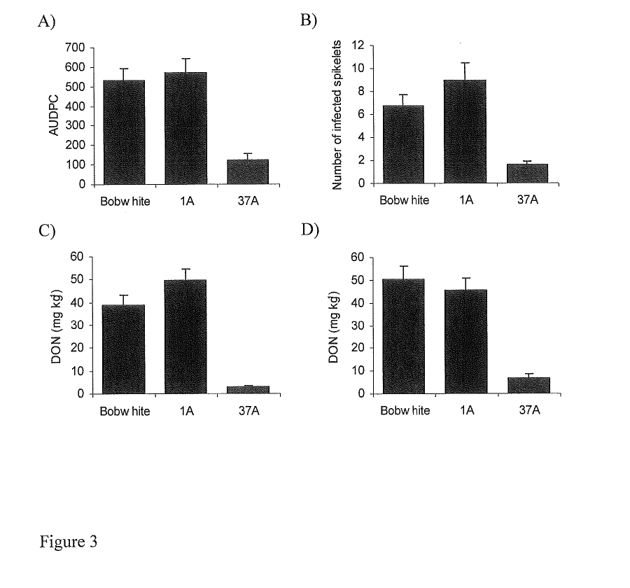

The spring wheat variety Bobwhite is moderately susceptible to FHB. RNA interference (RNAi)-induced gene silencing of Ein2 has been reported for this variety (Travella et al., 2006). The FHB resistance of Bobwhite was compared with that of two transgenic lines (A1 and A37) differing in their degree of Ein2 silencing. No significant gene silencing was detected in A1 while expression of Ein2 was reduced by approximately 50 percent (FHB resistance was assessed using both point and spray inoculation trials. The former assesses resistance to spread within the head, so-called Type 2 FHB resistance (sensu Schroeder and Christensen, 1963), while the latter assesses a combination of Type 1 (resistance to initial infection) and Type 2 resistance. For both types of inoculation ANOVA showed that the two trials did not differ significantly and the combined results are presented. Following spray inoculation (assessing the combined effects of resistance Types 1 and 2) Bobwhite and line A1 (non-silenced) did not differ significantly (P=0.68) for AUDPC (531 and 571 respectively). In contrast the AUDPC for the Ein2 gene-silenced line A37 (122), however, was significantly less that that in the Bobwhite parental line (P<0.001) (FIG. 3A). Similar results were obtained following point inoculation (assessing Type 2 resistance only) Bobwhite and line A1 (non-silenced) did not differ significantly (P=0.19) in the number of diseased spikelets (6.7 and 8.9 respectively) (FIG. 3B). The number of diseased spikelets on line A37 (1.6), however, was significantly less that that in the Bobwhite parental line (P<0.001) (FIG. 3B).

Attenuation of ET signalling through RNAi of Ein2 significantly reduced disease development following both spray and point inoculation under high disease pressure indicating that both Type 1 and Type 2 resistance is enhanced. Irrespective of the reduction in symptoms it is critical, from the food safety perspective, that this is associated with a reduction in mycotoxin accumulation (DON). Most strikingly accumulation of DON in grain of line A37 was reduced approximately 10 fold following both spray and point inoculation (FIGS. 3C and D). DON content of grain did not differ significantly (P=0.13) between Bobwhite and line A1 (39.0 and 49.6 mg kg⁻¹ respectively) while that for the Ein2 gene-silenced line A37 (3.2 mg kg⁻¹), was significantly less that that in the Bobwhite parental line (P<0.001) (FIG. 3C). Similarly, following point inoculation the DON content of grain harvested from Bobwhite and line A1 (50.3 and 45.5 3.2 mg kg⁻¹ respectively) did not differ significantly (P=0.56) while DON content of grain of line A37 (6.7 mg kg⁻¹), was significantly less that that in the Bobwhite parental line (P<0.001) (FIG. 3D). Attenuation of ET signalling reduced infection and spread of F. graminearum in wheat heads and, most importantly resulted in significantly reduced accumulation of DON mycotoxin in grain.

ET Signalling Plays a Role in DON-Induced Cell Death in Wheat Leaves

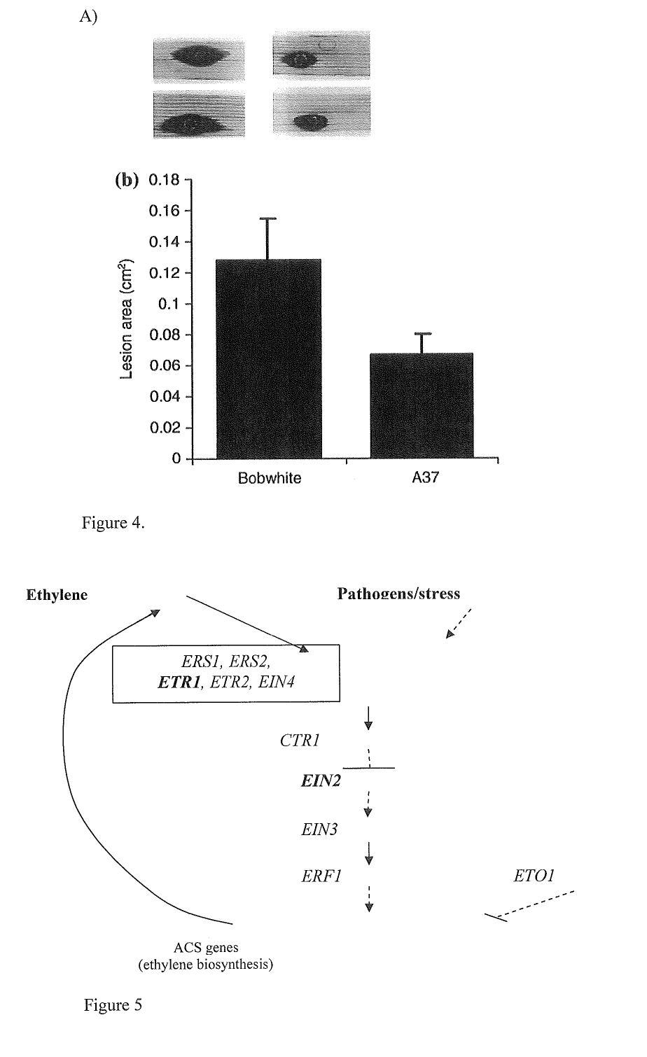

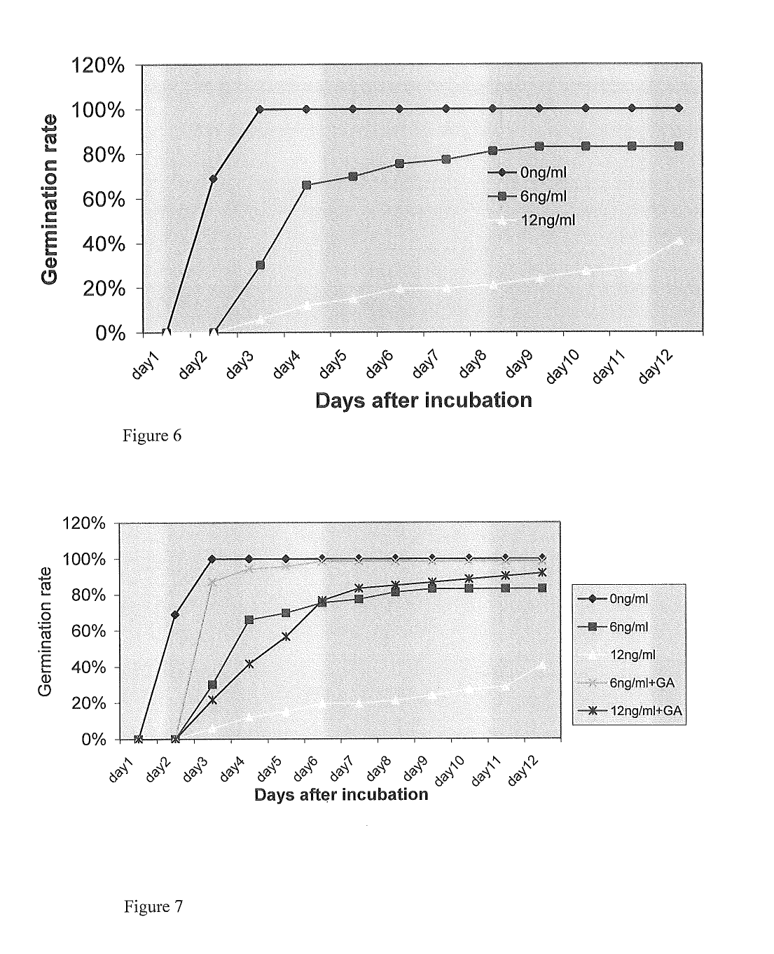

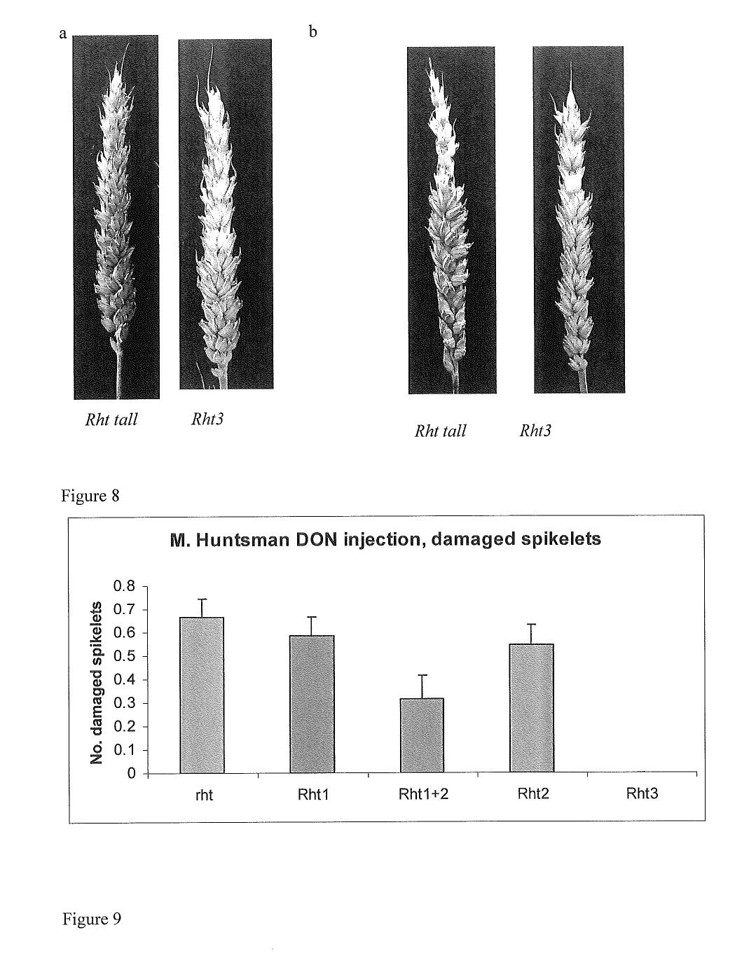

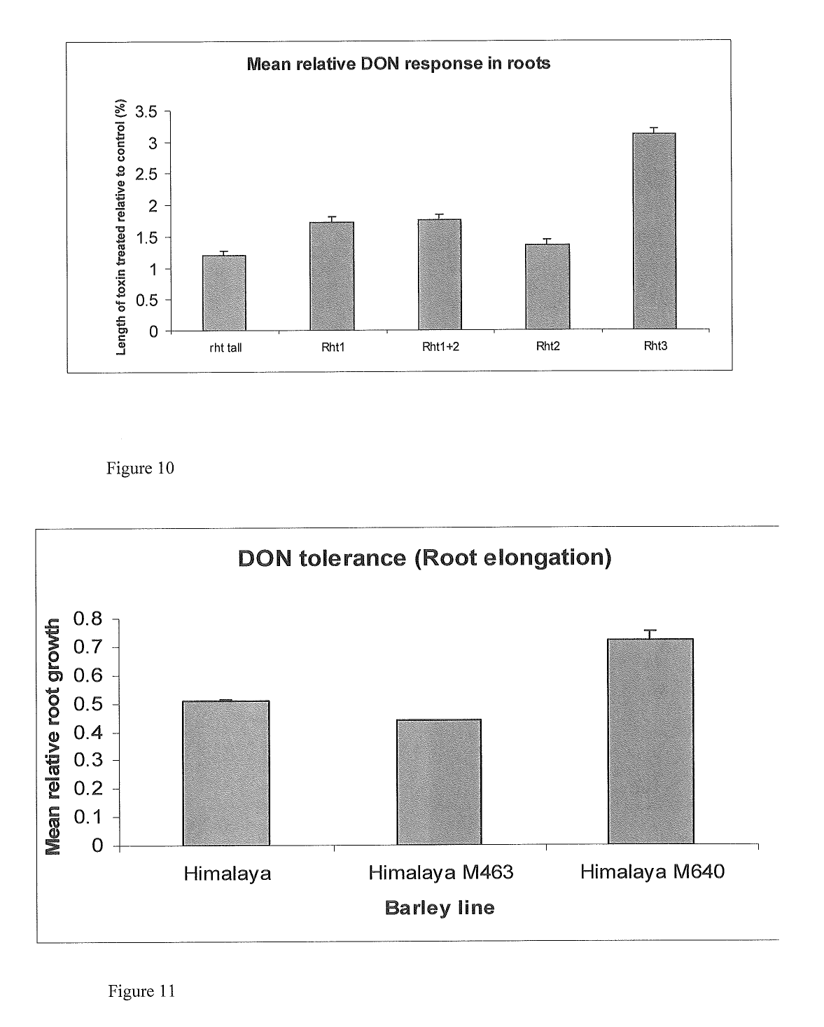

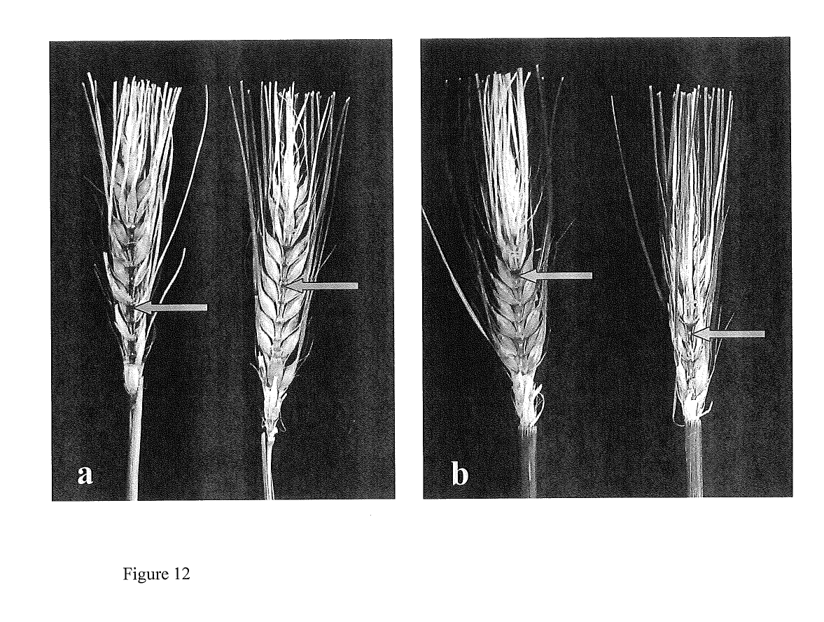

The above results showed that Ein2 plays an important role in susceptibility to spread of F. graminearum in wheat heads. Previous reports have shown that DON production by the fungus is required to enable spread between spikelets to occur (Bai et al., 2001; Jansen et al., 2005). We reasoned that DON may be functioning in part through ET signalling to achieve this effect. To test this we compared the effect of DON on leaves of Bobwhite and the Ein2 silenced line A37. Trypan blue staining revealed extensive cell death (FIG. 4A) about the inoculation point in both lines following exposure to DON (150 μMol). However, the size of lesions was significantly less (P<0.001) in A37 than in Bobwhite (0.066 and 0.127 cm² respectively) (FIG. 4B). These results indicate that reducing ET signalling leads to reduced DON-induced cell death in wheat leaves.

Previously, using a detached leaf bioassay, we demonstrated that F. graminearum is able to infect leaves of Arabidopsis. In the present study we showed that the ET signalling pathway is negatively associated with resistance to F. graminearum an that the outcome with respect to susceptibility is largely dependent upon ET biosynthesis, signal transduction and perception. Fusarium graminearum is regarded as a necrotroph and studies have not revealed any evidence for an initial biotrophic phase during colonisation of wheat heads (Jansen et al, 2005). The involvement of ET/JA signalling pathways in defence against necrotrophs has previously been well documented in studies with dicot species (Glazebrook, 2005).

Interestingly, in the interaction between Arabidopsis and F. graminearum we observed that disruption of the JA signal components COI1 and PAD1 increased susceptibility (results not shown). Thus our studies with F. graminearum reveal an additional permutation in which the ET and JA signalling pathways function with contrasting effects with respect to resistance to this pathogen. Signalling through the ET pathway acts negatively to increase susceptibility while the JA pathway functions positively in defence against F. graminearum. The contrasting effects on resistance to F. graminearum of mutations in genes associated with ET- and JA-related pathways may be a reflection of antagonistic interactions between ET and JA signalling such as described for responses to different types of stress and physiological processes. In the present study we observed that transcript accumulation of Vsp1 the JA specific gene in etr1-1 was strongly enhanced relative to that in wild-type lines following infection suggesting that ET signalling antagonizes the JA-dependent expression of VSP1.

The observation that a gain-of-function mutation in ACS5 (eto2) showed enhanced susceptibility implicates ACS5 in the response to F. graminearum. The fact that ACS5 is one of the eight functional members of ET biosynthesis ACS gene family suggests that different ACS isoforms may respond differentially to distinct elicitors and that ACS5 is perhaps activated specifically in response to F. graminearum. Mutation in ETO1, which is a negative regulator of ET biosynthesis that inhibits ACS enzyme activity and targets it for protein degradation (Wang, 2004), also showed enhanced susceptibility to F. graminearum. Significantly, ETO1 is thought to specifically and negatively interact only with a subset of ACS isoenzymes, the so-called type 2 class of which ASC5 is a member. It is possible that the greater susceptibility of eto1 and eto2 to F. graminearum is due to enhanced ET production following challenge by this pathogen. The role of ET in enhancing disease was further suggested by the responses to raised ET levels or interference of ET perception by silver ions. Our results also showed that, exogenous ET can promote disease symptoms independently of the downstream signal transduction pathway, but that the pathway itself is required for full susceptibility in the presence of exogenous ethylene. While ET production enhances susceptibility predominantly through the action of the characterised downstream signalling pathway ET also promotes disease development independently through an unknown pathway.

In contrast to the extensive literature on the involvement of different signalling pathways in interactions between pathogens and dicot species, very few studies have been carried out involving necrotrophic pathogens on monocot hosts. Our work with F. graminearum reveals that signalling through the host ET pathway acts to increase susceptibility in both dicot species such as Arabidopsis and monocot species such as wheat and barley. These results indicate that F. graminearum exploits ET signalling in both dicot and monocot species to aid colonisation. Mutations in Arabidopsis of components of ET biosynthesis, perception and signal transduction are all affected in their resistance to F. graminearum. Reduced disease symptoms in ET-insensitive mutants have also been observed in other studies of dicot hosts with a number of bacterial and fungal pathogens. For example, the Arabidopsis ET-insensitive mutant ein2 displayed reduced symptoms following infection by virulent strains of the bacterial pathogens P. syringae pv. tomato and pv. maculicola as well as X. campestris pv. campestris (Bent et al., 1992). Similarly in tomato, the ET-insensitive mutant Nr exhibited reduced symptoms when challenged with virulent strains of Fusarium oxysporum, Pseudomonas syringae pv. tomato, and Xanthomonas campestris pv. vesicatoria (Lund et al., 1998). The negative role of ET production and signal transduction in defence against F. graminearum is also similar to that found for interactions with the sugar beet cyst nematode H. schachtii (Wubben et al., 2001). The reduced symptoms observed in the bacterial studies are believed to reflect tolerance rather than resistance because the levels of bacterial growth in the mutants were similar to those in the wild type controls. The restricted disease symptoms following F. graminearum infection of ET biosynthesis and ET-insensitive mutants, however, represents true resistance because reduced disease symptoms in the mutants were always accompanied by reduced conidial production by F. graminearum.

It appears that F. graminearum modulates a cell death pathway as a strategy for host colonization, and that ethylene may play a central role in mediating F. graminearum induced cell death. There are several lines of evidence supporting this view, for example, infiltration of DON into leaves of Arabidopsis or wheat induces cell death and FIG. 4). In Arabidopsis leaves infected with F. graminearum extensive cell death occurs in host cells in advance of the extending hyphal tips, in cells adjacent to vascular tissues beyond the area colonised by the fungus and across secondary-infected leaves (Chen et al. 2006). Furthermore, DON-induced cell death in wheat was significantly less in the Ein2 silenced line than in the parental variety Bobwhite. ET is known to influence cell death in response to both biotic and abiotic stress factors. For instance, accumulation of 1-aminocyclopropane-1-carboxylic acid (ACC) and ET has been shown to be associated with cell death induced by AAL- and Fumonisin B1-toxin while inhibition of ET biosynthesis or signalling attenuates cell death induced by these toxins. In Arabidopsis protoplasts, Fumonisin B1-induced cell death was found to require ET, JA and SA pathways. Activation of the ET pathway in response to F. graminearum infection is suggested by the elevated expression of PDF1.2 and PR4 observed previously (Chen et al., 2006).

In the present study we have shown that the ET pathway is exploited by F. graminearum to aid colonisation of Arabidopsis and wheat. Our results also suggest that DON, which is an acknowledged virulence factor on wheat may function through an ET mediated signal to induce cell death. Thus it appears that plant pathogens may manipulate or exploit plant hormone homeostasis to suppress or overcome defence responses.

In conclusion, we have used an Arabidopsis bioassay to reveal insights into interactions between plant hosts and an important pathogen of cereals. We have then undertaken chemical and genetic studies on cereal hosts and provided an example of translation from a dicot model to a monocot crop host. We have shown that attenuation of ET signalling in wheat can significantly reduce FHB symptom development, but most importantly, this has a proportionately greater effect on DON accumulation in grain. We propose that appropriate manipulation of ET synthesis/signalling/perception in wheat and barley may provide a means to increase FHB resistance and reduce the risk posed to consumers of mycotoxins accumulating in grain.

### Example 2

### The Effect of Gibberellin on Pathogen Resistance

We have been combining genetic analyses of wheat and barley with studies involving Arabidopsis in order to investigate mechanisms involved in resistance/susceptibility to FHB and DON. The results from these studies have demonstrated the importance of two phytohormone signalling pathways in FHB. Several authors have reported a negative relationship between plant height and FHB resistance (Miedaner, 1997; Buerstmayr et al., 2000; Somers et al., 2003). We recently reported a potent FHB resistance QTL coincident with the Rht-D1 plant height (PH) locus in wheat segregating in a cross between Arina that carries the Rht-D1a allele and Riband that has the Rht-D1b allele, also known as Rht2 (Draeger et al., 2007). Results from the Arina×Riband population suggested that the relationship between plant height and FHB susceptibility is not due to plant height per se but, rather to either linked genes conferring FHB susceptibility and/or a pleiotropic physiological effect of the Rht-D1b allele enhancing susceptibility. Rht-D1b, carried on chromosome 4D is found in most UK winter wheat varieties and the allele responsible for their semi-dwarf stature. A second semi-dwarfing allele, Rht-B1b (Rht1) is homoeologous to Rht-D1 and is carried on chromosome 4B. Both Rht-B1 and Rht-D1 encode so-called DELLA proteins that are negative regulators of gibberellin (GA) signalling. The semi-dwarf ‘1b’ alleles encode stabilised versions of the DELLA proteins resulting in GA-insensitive semi-dwarf plants while the ‘1c’ alleles encode proteins with even greater stability resulting in extremely dwarfed plants. We have undertaken numerous studies involving populations with parents that differ in Rht status and near-isogenic Rht tall, semi-dwarf and dwarf lines to investigate the relationship between FHB susceptibility and Rht.

Materials and Methods

We have tested Huntsman isolines by point inoculation and also for DON tolerance. These included (with the degree of dwarfing and chromosome location)
Rht1—moderate effect 4B
Rht3—severe effect 4B
Rht2—moderate effect 4D
Rht10—severe effect 4D
rht—tall

We also used a Petritox assay of GA-insensitive (M640) and GA-sensitive (M463) barley mutants of variety Himalaya. This test is used to study toxin tolerance on a toxin (DON, 3-AcDON)-containing medium.

Results

Using point inoculation experiments, we showed that Rht3 confers a significant resistance to F. culmorum (DON-producer) spread and to treatment with DON mycotoxin. Spreading of necrosis in Rht lines was reduced. Two of the most resistant varieties grown in the UK are Spark, (Rht-D1a) and Soissons (Rht-B1b). We undertook QTL analyses of a population segregating for Rht-D1a (Spark) and Rht-D1b (Rialto) and a population derived from Soissons (Rht-B1b) and Orvantis (Rht-D1b). A stable QTL was observed in both populations at the Rht-D1 locus across diverse environments with susceptibility being associated with the Rht-D1b allele (Srinivasachary et al., 2008). Surprisingly, no similar effect was seen for the Rht-B1 locus, and in one trial the Rht-B1b allele (contributed by Soissons) even conferred a very minor positive effect. The effect of the Rht-B1 and Rht-D1 loci on FHB susceptibility was further examined in a range of experiments involving near-isogenic lines in Mercia and Maris Huntsman differing for alleles at the Rht loci. Under high disease pressure both Rht-B1b and Rht-D1b significantly decreased Type 1 resistance (resistance to initial infection). However, while Rht-D1b had no effect on Type 2 resistance (resistance to spread of the fungus within the spike), Rht-B1b significantly increased Type 2 resistance. The majority of UK winter wheat varieties are highly susceptible to FHB and almost all these carry the semi-dwarfing Rht-D1b allele (Gosman et al., 2007). Neither Soissons nor Spark carry Rht-D1b: Soissons possesses Rht-B1b and Spark has a tall (rht) genotype with its reduced height being due to non-Rht genes. It appears that the difference in FHB resistance between these two varieties and the others on the UK National List of 2003 may, in large part, be simply a reflection of the presence or absence of Rht-D1b. Under conditions of moderate disease pressure, use of the Rht-B1b semi-dwarfing allele may provide the desired crop height without compromising resistance to FHB to the same extent as lines carrying Rht-D1b.

It was not possible, on the basis of these results to resolve whether these effects were due to pleiotropy or linkage to genes that differed in their effect on FHB resistance. Additional experiments were undertaken involving Rht-B1c and Rht-D1c near-isogenic lines and Rht mutant lines of the barley variety Himalaya. The results clearly showed that mutation of Rht increased Type 2 resistance and that the effect was more pronounced for dwarf (‘c’ alleles) than semi-dwarf lines (‘1b’ alleles). Further experiments involving DON were carried out on leaves and germinating grain. Again, the results clearly showed that mutation of the DELLA genes increased resistance to DON. We conclude that interference with GA signalling enhances resistance to DON and, as a consequence, leads to enhanced Type 2 resistance. Lines carrying Rht-D1b, however, do not conform to this model and we, currently, believe that this is due to tight linkage of this allele to a gene reducing Type 2 resistance and so enhancing susceptibility to FHB. In summary, GA-insensitivity (DELLA mutation) appears to confer type 2 resistance to FHB (wheat and barley); GA-insensitivity (DELLA mutation) appears to confer resistance to DON as assayed by root elongation and application to ears (wheat and barley); Rht3 and, to a lesser extent Rht1 confers resistance to FHB and DON. The FHB/DON resistance is greater for the more potent Rht allele; DON enhances expression of Rht genes in ‘tall’ wheat lines but less/not at all in Rht mutants.

### REFERENCES

Bai, G.-H., Desjardins, A. E., Plattner, R. D. 2001. Deoxynivalenol-non producing Fusarium graminearum causes initial infection, but does not cause disease spread in wheat spikes. Mycopathologia 153: 91-98.
Bent et al Disease and Performance-Related Traits of Ethylene-Insensitive Soybean Published in Crop Sci. 46:893-901 (2006)
Bent, A. F., R. W. Innes, et al. (1992). Disease development in ethylene-insensitive Arabidopsis thaliana infected with virulent and avirulent Pseudomonas and Xanthomonas pathogens. Mol Plant Microbe Interact 5(5): 372-8.
Berrocal-Lobo, M., Molina, A. (2004). ETHYLENE-RESPONSE-FACTORI mediates Arabidopsis resistance to the soilborne fungus Fusarium oxysporum. Mol Plant Microbe Interact 17(7): 763-70.
Berrocal-Lobo, M., Molina, A., Solano, R. (2002). Constitutive expression of ETHYLENE-RESPONSE-FACTORI in Arabidopsis confers resistance to several necrotrophic fungi. Plant Journal 29(1): 23-32.
Bleecker et al The Plant Cell, Vol. 18,3347-3349, December 2006
Buerstmayr, H., Steiner, B., Lemmens, M., Ruckenbauer, P. 2000. Resistance to Fusarium head blight in winter wheat: heritability and trait associations. Crop Science 40: 1012-1018.
Chang et al 2004 Ethylene Biology. More Than a Gas Plant Physiology, Vol. 136, pp. 2895-2899
Chen, X., Steed, A., Harden, C., and Nicholson, P. (2006). Characterisation of Arabidopsis thaliana-Fusarium graminearum interactions and identification of variation in resistance among ecotypes. Molecular Plant Pathology 7.
Cuthbert P A, Somers D J, Brule-Babel A (2007) Mapping of Fhb2 on chromosome 6BS: a gene controlling Fusarium head blight field resistance in bread wheat (Triticum aestivum L.). Theor Appl Genet 114:429-437
Desjardins, A. E., Proctor, R. H., Bai, G., McCormick, S. P., Shaner, G., Beuchley, G., Hohn, T. M. 1996. Reduced virulence of trichothecenes antibiotic-nonproducing mutants of Gibberella zeae in wheat field tests. Molecular Plant-Microbe Interaction 9: 775-781.
Diaz, J., Have, A., and van Kan, J. A. L. (2002). The Role of Ethylene and Wound Signalling in Resistance of Tomato to Botrytis cinerea. Plant Physiology 129: 1341-1351.
Draeger, R., Gosman, N., Steed, A., Chandler, E., Thomsett, M., Srinivasachary, Schondelmaier, J., Buerstmayr, H., Lemmens, M., Schmolke, M., Mesterhazy, A., Nicholson P. 2007. Identification of QTL for resistance to Fusarium head blight, DON accumulation and associated traits in the winter wheat variety Arina. Theoretical and Applied Genetics, 115: 617-625.
Feys, B. J., Parker, J. E. (2000). Interplay of signalling pathways in plant disease resistance. Trends Genet 16(10): 449-55.
Fleck B, Harberd N P (2002) Evidence that the Arabidopsis nuclear gibberellin signalling protein GAI is not destabilized by gibberellin. Plant J 32: 935-947
Geraats, B. P. J., Bakker, P. A. H. M., Linthorst, H. J. M., Hoekstra, J., van Loon, L. C. (2007) The enhanced disease susceptibility phenotype of ethylene-insensitive tobacco cannot be counteracted by inducing resistance or application of bacterial antagonists. Physiological and Molecular Plant Pathology 70: 77-87.
Gilbert, J., Tekauz, A. 2000. Review: Recent developments in research on fusarium head blight of wheat in Canada. Canadian Journal of Plant Pathology 22: 1-8.
Glazebrook, J. (2005) Contrasting mechanisms of defence against biotrophic and necrotrophic pathogens. Annual Review of Phytopatholoy 43: 205-227.
Gosman, N., Bayles, R., Jennings, P., Kirby, J., Nicholson, P. 2007. Evaluation and characterization of resistance to Fusarium head blight caused by Fusarium culmorum in UK winter wheat cultivars. Plant Pathology 56: 264-276.
Grant, M., Lamb, C., (2006) Systemic immunity. Current Opinion in Plant Biology 9: 414-420.
Guo and Ecker The ethylene signalling pathway: new insights, Current Opinion in Plant Biology, Volume 7, Issue 1, February 2004, Pages 40-49
Heil et al Induced Systemic Resistance (ISR) Against Pathogens in the Context of Induced Plant Defences, Annals of Botany 89: 503-512, 2002
Jansen C, von Wettstein D, Schafer W, Kogel K H, Felk A, Maier F J, 2005. Infection patterns in barley and wheat spikes inoculated with wild-type and trichodiene synthase gene disrupted Fusarium graminearum. Proceedings of the National Academy of Sciences of the United States of America 102, 16892-16897.
Lund, S., Stall, R E, Klee, H J. (1998). Ethylene regulates the susceptible response to pathogen infection in tomato. Plant Cell 10: 371-382.
Miedaner, T. 1997. Breeding wheat and rye for resistance to Fusarium diseases. Plant Breeding 116: 201-220.
O'Donnell, P. J., Schmelz, E. A., Moussatche, P., Lund, S. T., Jones, J. B., Klee, H. J. (2003). Susceptible to intolerance—a range of hormonal actions in a susceptible Arabidopsis pathogen response. Plant J 33(2): 245-57.
O'Donnell, K., Ward T. J., Geiser, D. M., Kistler, H. C., Aoki, T., 2004. Genealogical concordance between the mating type locus and seven other nuclear genes supports formal recognition of nine phylogenetically distinct species within the Fusarium graminearum Glade. Fungal Genetics and Biology 41:600-623.
Olszewski 2002. Gibberellin Signalling: Biosynthesis, Catabolism, and Response Pathways. The Plant Cell Supplement, S61-S80
Okushima et al 2005, Plant Journal 443: 29-46
Peng et al 1999 ‘Green Revolution’ genes encode mutant gibberellin response modulators. Nature 400: 256-261
Placinta, C. M., D'Mello, J. P. F., Macdonald, A. M. C. 1999. A review of worldwide contamination of cereal grains and animal feed with Fusarium mycotoxins. Animal Feed Science and Technology 78: 21-37.
Richards et al 2001 How gibberellin regulates plant growth and development: a molecular genetic analysis of gibberellin signalling. Annu Rev Plant Physiol Plant Mol Biol 52:67-88
Srinivasachary, Gosman, N., Steed, A., Simmonds, J., Leverington-Waite, M., Wang, Y., Snape, J., Nicholson, P. 2008. Susceptibility to Fusarium head blight is associated with the Rht-D1b semi-dwarfing allele in wheat. Theoretical and Applied Genetics 116: 1145-1153.
Schroeder H W, Christensen J J, 1963. Factors Affecting Resistance of Wheat to Scab Caused by Gibberella zeae. Phytopathology 53, 831-838.
Shen X, Zhou M, Lu W, Ohm H, 2003. Detection of Fusarium head blight resistance QTL in a wheat population using bulked segregant analysis. Theoretical and Applied Genetics 106, 1041-1047.
Singh, M. P., Lee, F. N., Counce, P. A., Gibbons, J. H. (2004) Mediation of partial resistance to rice blast through anaerobic induction of ethylene. Phytopathology 94:819-825.
Singh, M. P.; Lee, F. N.; Counce, P. A. (2003) Role of ethylene in rice blast field resistance. Phytopathology 93 (6 Supplement):S79.Schroeder, H. W., Christensen, J. J. 1963. Factors affecting resistance of wheat to scab caused by Gibberella zeae. Phytopathology 53: 831-837.
Somers, D. J., Fedak, G., Savard, M. 2003. Molecular mapping of novel genes controlling Fusarium head blight resistance and deoxynivalenol accumulation in spring wheat. Genome 46: 555-564.
Steed, A., Chandler, E., Thomsett, M., Gosman, N., Faure, S., Nicholson, P. (2005) Identification of Type 1 resistance to Fusarium head blight controlled by a major gene located on chromosome 4A of Triticum macha. Theoretical and Applied Genetics 111: 521-529.
Thomas et al 2004 Update on Gibberellin Signalling. A Tale of the Tall and the Short Plant Physiology, Vol. 135, pp. 668-676
Travella et al 2006. RNA Interference based gene silencing as an efficient tool for functional genomics in hexaploid bread wheat. Plant Physiology, 142, 6-20
Van Loon et al, Ethylene as a modulator of disease resistance in plants, Trends in Plant Science, 11 (4), 2006, 184-190
Van Wees, S., De Swart, E., Van Pelt, J., Van Loon, L. and Pieterse, C. (2000). Enhancement of induced disease resistance by simultaneous activation of salicylate- and jasmonate-dependent defense pathways in Arabidopsis thaliana. Proc. Natl. Acad. Sci. USA, 97: 8711-8716.
Van Wees, S. C., Chang, H. S., Zhu, T., Glazebrook, J. (2003). Characterization of the early response of Arabidopsis to Alternaria brassicicola infection using expression profiling. Plant Physiol 132(2): 606-17.
Wang et al Regulation of ethylene gas biosynthesis by the Arabidopsis ETO1 protein Nature. 2004 Apr. 29; 428(6986): 945-50
Wubben, M. J., Su, H., Rodermel, S. R., Baum, T. J. (2001). Susceptibility to the sugar beet cyst nematode is modulated by ethylene signal transduction in Arabidopsis thaliana. Mol Plant Microbe Interact 14(10): 1206-12.

Sequence Listing

EIN2
SEQ ID No. 1
caccaaagcccaaattgcatttaatgagcgctcacaacataatctccaa
agagatgtcctttctatgcagttgggtatgaaccccaacaataaatccc
tttgggcccaacaaccgtttgaacagctgtttggtatgtcaagtgcaga
actgaataagagtgaggtgaacactggccagagatcaagtggcatgaca
aaggatgattcctcatacacagagtgtgaggcagagcttcttcaatctc
ttaggctttgcataatgaatatcttgaaactggaaggatcaggagggct
ctagg
barley ARF2
(used for silencing construct)
SEQ ID No. 2
caccaaaggatctgcatggcatggactggcgcttccgccatatcttccg
tggtaagtttctgttccgtgccttgctctgatctgtggcagttttacat
ccccatgtatgcccagtgtgtgtgatctgaagctgataacttcagtaga
ccatttggttgtagcttgcaatcagtgacaccgcaacacatcaaatctg
cctataaattgcaagggtttatcttgtacttgatgatggtgatggcacc
gatgataattgtgttaaccgagtgcaaatcaacagggcaacctaggagg
catctccttcagagcggttggagtgtgtttgtcagttccaaaaggcttg
tagctggggatgccttcattttcctcaggtctgttgctgtttccttact
caccagcatagcaattcttaacctggtgctaaatgtgttctgctccaca
cagaggagagagtggcgagcttcgtgttggtgttaggcgggctatgaga
cagctgtccaacgtgccttcttcagtcatttctagtcatagcatgcatc
ttggggtccttgcaactgcatggcacgctatcaacacgaaaagcatgtt
cacggtctactacaaacctaggtacatcaacaatgctaaggcaatcatg
cccttctatatgtagtcattaatttgttcctggtggctcattctgagta
cttacactactgtctaatctttggtcgattttagaacgagcccttcaga
gttcattataccatatgatcaatatatggagtctgtgaagaacaactat
caattgggatgagattcaggatgaggtttgaaggcgaagaggcaccaga
gcaaaggtgactgtcgtaattgcttttcctacaagtgtagtttggtgtg
catgatcccccaacagcaccgagtagatcatttctaatttgctgtttcc
attgtcatataggtttactggtactatagttggcagtgaaa
Deduced amino acid coding sequence
for ARF2 within the barley silencing construct
(deduced by comparison with the ARF2 sequence
for rice
(BAB85913.1/GI:19352039)
SEQ ID No. 3
KDLHGMDWRFRHIFRGQPRRHLLQSGWSVFVSSKRLVAGDAFIFLRGES
GELRVGVRRAMRQLSNVPSSVISSHSMHLGVLATAWHAINTKSMFTVYY
KPRTSPSEFIIPYDQYMESVKNNYSIGMRFRMRFEGEEAPEQR

1. A method for conferring resistance to Fusarium Head Blight (FHB) to a plant, the method comprising decreasing the production of a plant hormone in the plant or reducing the responsiveness of the plant to a plant hormone, wherein the plant hormone is ethylene or gibberellin.

2. The method of claim 1, wherein the plant hormone is ethylene.

3. The method of claim 1, wherein the FHB is F. culmorum, F. graminearum, F. avenaceum, F. poae, F. asiaticum or Gibberella zeae.

4.-10. (canceled)

11. The method of claim 2, wherein production of ethylene is decreased by silencing or mutating one or more genes encoding a component of the ethylene biosynthesis pathway.

12. The method of claim 11, wherein the one or more genes encode ACS or ACO or an orthologue or homolog thereof.

13. The method of claim 2, wherein production of ethylene is decreased by exposing the plant to an agent that reduces ethylene production.

14.-16. (canceled)

17. The method of claim 2, wherein the responsiveness of the plant to ethylene is decreased by silencing or mutating one or more genes encoding a component of the ethylene signalling pathway.

18.-20. (canceled)

21. The method of claim 17, wherein the component of the ethylene signalling pathway is a receptor or transcription factor.

22. The method of claim 17, wherein the one or more genes encoding a component of the ethylene signalling pathway are selected from the group consisting of ETR1, ETR2, ERS1, ERS2, EIN2, EIN3, EIN4, EIN5, EIN6, EIL1, CTR and an orthologue or homolog thereof.

23.-27. (canceled)

28. A method of reducing the presence of mycotoxins in a plant the method comprising decreasing the production of ethylene in the plant or reducing the responsiveness of the plant to ethylene.

29.-30. (canceled)

31. A method for producing a plant with increased resistance to FHB, the method comprising mutagenizing one or more genes encoding a component of the ethylene production or ethylene signalling pathway.

32. (canceled)

33. A method for of screening plants which are resistant to FHB, the method comprising identifying a plant with reduced ethylene production, reduced responsiveness to ethylene, reduced gibberellin production, and/or reduced responsiveness of gibberellin.

34.-38. (canceled)

39. The method of claim 1, wherein the plant hormone is gibberellin.

40. The method of claim 39, wherein the FHB is F. culmorum, F. graminearum, F. avenaceum, F. poae, F. asiaticum or Gibberella zeae.

41.-45. (canceled)

46. The method of claim 39, wherein gibberellin production is decreased by mutating or silencing one or more of the genes encoding a component of the gibberellin biosynthesis pathway.

47. (canceled)

48. The method of claim 46, wherein the one or more genes encode one or more of the following enzymes: copalyl diphosphate synthase; ent-kaurene synthase; Dwarf3; gibberellin 20-oxidase; gibberellin 7-oxidase; gibberellin 3 [beta]-hydroxylase; and ent-kaurene oxidase or an orthologue or homolog thereof.

49. The method of cliam 39, wherein production of giberrellin is decreased by exposing the plant to an agent that reduces gibberellin production.

50. The method of claim 49, wherein the agent is trinexapac-ethyl, flurprimidol, paclobutrazol or a combination thereof.

51. The method of claim 39, wherein reducing the responsiveness of the plant to gibberellin comprising mutating or silencing one or more genes encoding a component of the gibberellin signalling pathway.

52.-53. (canceled)

54. The method of claim 51, wherein the component of the gibberellin signalling pathway is a receptor or transcription factor.

55.-57. (canceled)

58. A method for producing a plant with increased resistance to FHB, the method comprising mutagenizing one or more genes encoding a component of the gibberellin production or gibberellin signalling pathway.

59.-67. (canceled)

68. A transgenic plant with increased resistance to FHB, wherein the plant produces reduced levels of ethylene or gibberellin or has a mutation in the signalling pathway responsive to ethylene or gibberellin.

69. A method for conferring resistance to FHB to a plant, the method comprising generating a transgenic plant comprising a mutation in the gene expressing the auxin response factor 2 or a functionally silenced gene expressing the auxin response factor 2.
